(19) United States
(12) Patent Application Publication
(10) Pub. No.: US 2008/0255442 A1
(43) Pub. Date: Oct. 16, 2008

(54) REGISTRATION SYSTEM AND METHOD

(76) Inventors: Alan Ashby, York (GB); Thorsten Burger, Munchen (DE); Marcellino Maheson, Pentwyn (GB)

Correspondence Address: Philip S. Johnson, Esq.;Johnson & Johnson, One Johnson & Johnson Plaza, New Brunswick, NJ, 08933-7003, US

(21) Appl. No.: 11/868,615

(22) Filed: Oct. 8, 2007

(30) Foreign Application Priority Data
Apr. 6, 2005 (GB) 05252143.2
Apr. 5, 2006 (GB) PCT/GB2006/001255

(51) Int. Cl. A61B 5/00

(52) U.S. Cl. 600/407

(57) ABSTRACT

Methods and systems for registering the position of a body part of a patient with a computer aided surgery system are described. A computer implemented method includes determining the position of a plurality of points of the body part, fitting the points to a representation of the body part, determining a closeness of fit for each of the points and displaying an indication of the closeness of fit. The system includes a tracking system for determining the position of an instrument engageable with a plurality of points of the body part and a control system. The control system is configured to determine the position of the plurality of points of the body part, fit the points to a representation of the body part, determine a closeness of fit for each of the points and display an indication of the closeness of fit for at least one of the points.

The present invention relates to systems and methods for use in registering a patient, and in particular to apparatus, methods computer program code and computer program products for intra-operatively registering a body part of a patient with a computer aided surgery system.

A feature of many computer aided surgery systems and methods is registration of the physical position of the patient, or a body part of the patient, in an actual physical reference frame, or co-ordinate system, of the world, with a reference frame or co-ordinate system of the computer aided surgery (CAS) system. Various techniques can be used which can depend on a number of factors, such as the stage in the surgical procedure (e.g. pre-operative, intra-operative) and the type of equipment or apparatus available (e.g. imaging systems such as ultrasound, X-ray, CT and similar).

One method can involve digitising a body part by applying the tip of a pointer device, or instrument, to various positions on the surface of the body part and capturing those positions using a tracking system which can determine the position of the pointer. This can be carried out percutaneously, which does not require any incisions, or subcutaneously, which generally will require at least one incision.

Irrespective, typically a large number of positions on the surface of the body part are captured in order to allow the position of the body part to be accurately registered with the CAS system and, for example, to be brought into registration with a previously captured image of the body part. It can take a significant amount of time to capture a large number of positions and the registration process can fail which requires the practitioner to re-capture all the positions. This can lead to delays in the surgical procedure and can also increase the risk of infection when there is an open incision.

Therefore, it would be advantageous to be able to provide a more efficient method for reliably registering a patient's body part.

According to a first aspect of the present invention, there is provided a computer implemented method for registering the position of a body part of a patient with a computer aided surgery system, comprising determining the position of a plurality of points of the body part, fitting the plurality of points to a representation of the body part, determining a closeness of fit for each of the plurality of points, and displaying an indication of the closeness of fit for at least one of the plurality of points.

Displaying an indication of the closeness of fit can include displaying the indication at the determined position of the point overlaid on an image of the body part at a fitted position of the image of the body part.

The indication can be a visual indication. The visual indication can be a colour. The colour can vary depending on the closeness of fit. Different shades, tones, intensities or other variations in colour can be used. A gray scale or spectrum or range of colours can be used. Different shapes or sizes of indicia can be used as the visual indication

A subset of points exceeding a closeness of fit can be displayed. The subset can correspond to points have a closeness metric exceeding a threshold value. The threshold value can be approximately 2 mm, 1.5 mm or 1 mm.

An indication of the closeness of fit can be displayed for each of a subset of the plurality of points An indication of the closeness of fit can be displayed for each of all of the plurality of points.

The method can further comprise re-determining the position of at least a one of the plurality of points and re-fitting those points of the plurality of points whose position has not been re-determined and those points whose position has been re-determined.

The plurality of points can include a set of landmark points and a set of surface points. The positions of the landmark points can be used to determine the direction and/or the size and/or the orientation of the body part. The positions of the surface points can be used to fit the positions of the plurality of points to the representation of the body part.

The representation of the body part can be a captured image of the body part or derived from a captured image of the body part. The representation of the body part can be a model of, or virtual, body part or derived from a virtual or model body part.

The method can further comprise assessing the spatial distribution of the closeness of fit of the plurality of points to identify at least a one of the plurality of points causing an unsuccessful fit.

The method can include using the closeness of fit of at least one anatomical landmark point to provide a visual indication to recapture the landmark point. One of the plurality of points can be a landmark point, and the closeness of fit of the landmark point can be determined. If the closeness of fit of the landmark point exceeds a threshold, then a visual indication to recapture the landmark point can be displayed.

The method can further comprise re-determining the position of the landmark point. The landmark point whose position has been re-determined can be used in re-fitting. All or some of those of the plurality of points whose position has not been re-determined can also be used in re-fitting. Preferably, at least the re-determined landmark point, a further landmark point whose position has not been re-determined and/or a plurality of surface points whose positions have not been re-determined are used in the re-fitting.

According to a further aspect of the invention, there is provided a computer aided surgery system for registering the position of a body part of a patient comprising a tracking system for determining the position of an instrument engageable with a plurality of points of the body part and a control system configured to determine the position of the plurality of points of the body part, fit the plurality of points to a representation of the body part, determine a closeness of fit for each of the plurality of points and display an indication of the closeness of fit for at least one of the plurality of points on a display device.

According to a further aspect of the invention, there is provided a method for registering the position of a body part of a patient with a computer aided surgery system, comprising identifying the position of a plurality of points of the body part with an instrument trackable by a tracking system, determining whether to re-identify the position of at least a one of the plurality of points from a display of an indication of the closeness of fit for at least one of the plurality of points, the closeness of fit having been determined for each of the plurality of points from a fit of the plurality of points to a representation of the body part and re-identifying the position of a subset of the plurality of points of the body part the subset comprising fewer points than all of the plurality of points.

According to further aspects of the invention there are provided computer program code executable by a data processing device to provide the method aspect of the invention and the system aspect of the invention. A computer program product comprising a computer readable medium bearing such computer program code can be a further aspect of the invention.

An embodiment of the invention will now be described, by way of example only, and with reference to the accompanying drawings, in which:

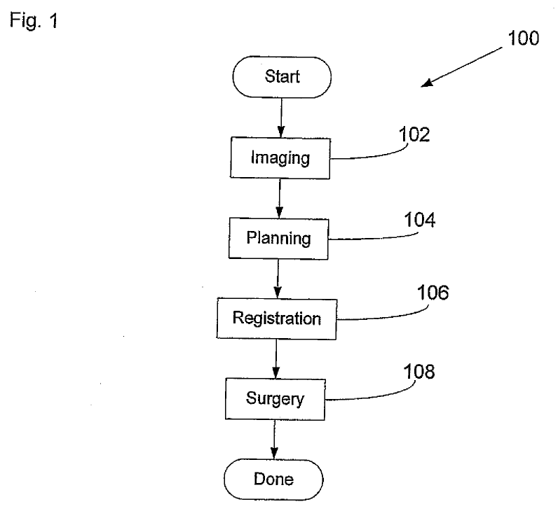

FIG. 1 shows a flow chart illustrating at a high level a computer aided surgical method in which the invention can be used;

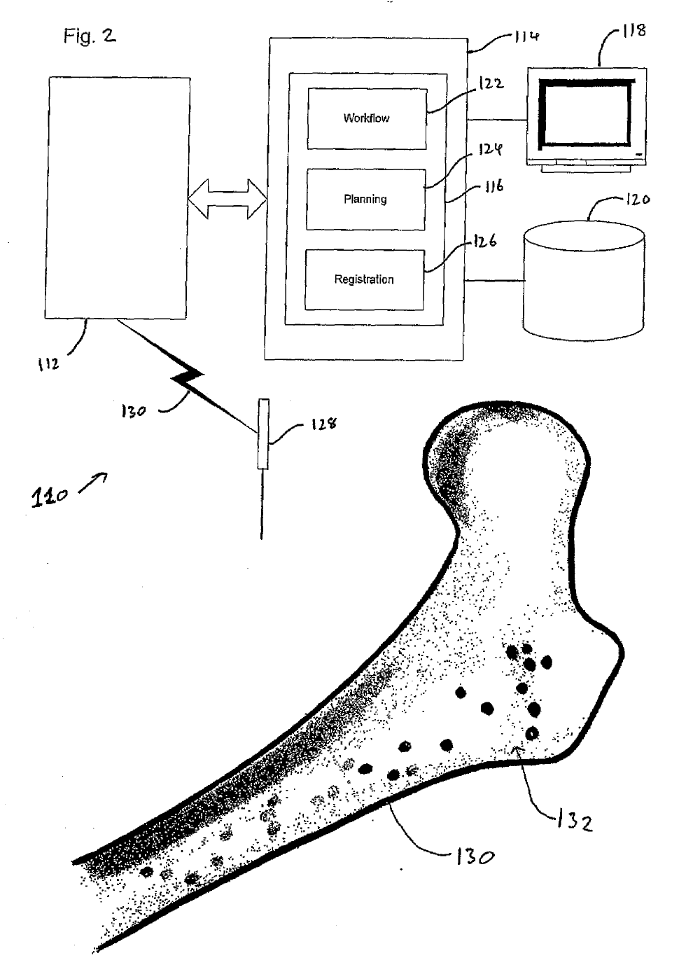

FIG. 2 shows a schematic block diagram of a computer aided surgery system according to the invention;

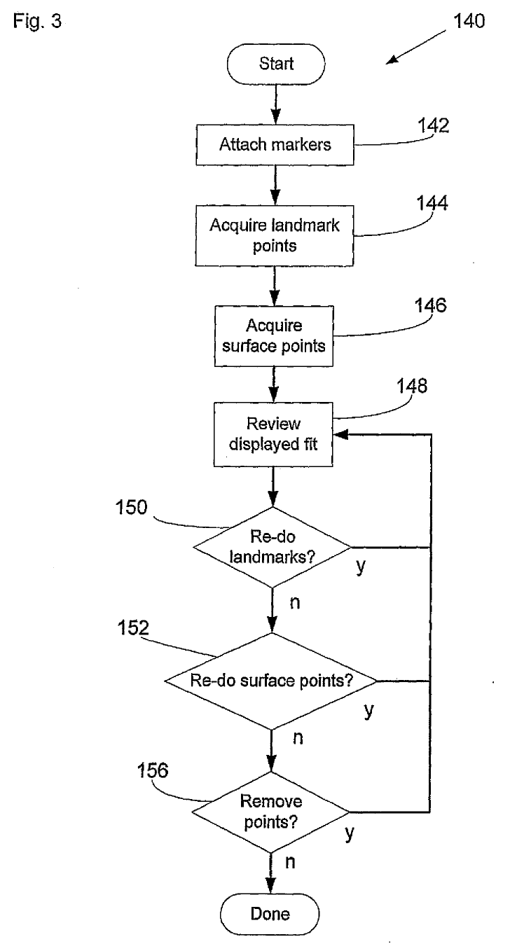

FIG. 3 shows a flow chart illustrating a registration method according to the invention;

FIG. 4 shows a flow chart illustrating a computer implemented registration method according to the invention;

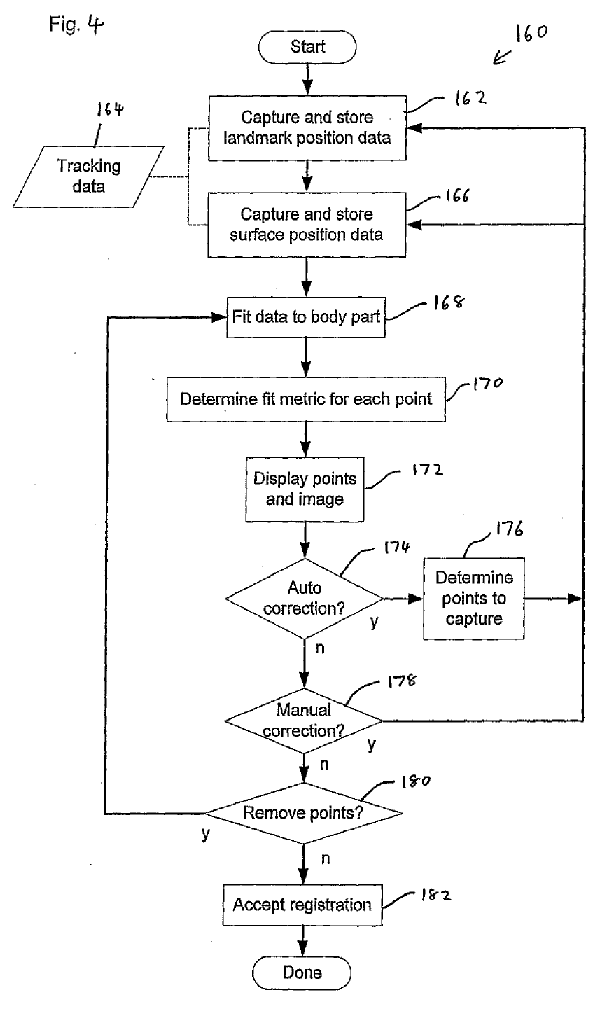

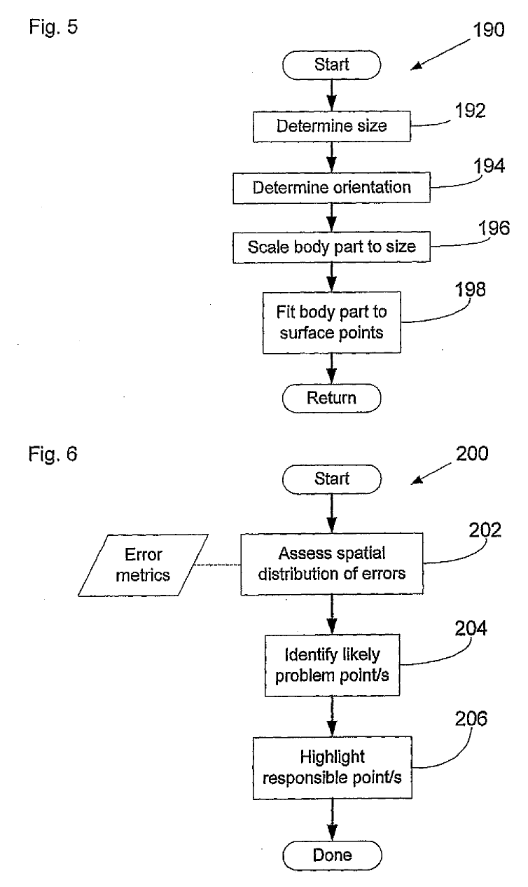

FIG. 5 shows a flow chart illustrating a fitting process part of the method illustrated in FIG. 4 in greater detail;

FIG. 6 shows a flow chart illustrating an auto correction process part of the method illustrated in FIG. 4 in greater detail; and

FIG. 7 shows a schematic block diagram illustrating a computer part of the system shown in FIG. 2.

Similar items in different Figures share common reference numerals unless indicated otherwise.

With reference to FIG. 1, there is shown a flow chart illustrating, at a high level a general surgical method 100 in which the registration method of the present invention can be used. The flow chart is schematic in that a number of actual actions carried out by a medical practitioner or practitioner may not strictly fall within a single one of the method steps illustrated and/or the order to sequence of the method steps may be varied. The flow chart is merely intended to help explain some of the actions that may be carried out and to provide context as to how the method of the invention can be utilised in practice.

Some of the actions, such as capturing images of the patient and surgical planing can be carried out pre-operatively, and either in the operating theatre or else where, in another medical or clinical facility, or intra-operatively in the operating theatre. In one embodiment the registration procedure can be carried out pre-operatively, or, in another embodiment, intra-operatively. The surgical procedure will generally be a computer aided surgical procedure and can be an image guided surgical procedure.

The present invention will be described in the exemplary context of an orthopaedic computer aided surgical procedure, and, in particular, a hip replacement surgical procedure. However, it will be appreciated that the utility of the present invention is not limited to that specific surgical procedure nor to orthopaedic procedures only. Rather, the invention can be of utility in any surgical, medical, clinical or diagnostic procedure in which registration of the body of a patient, or a part of the body of a patient, is beneficial.

Step 102 is an optional step in which an image or images of the body part of the patient are captured, processed and stored. Patient body images can be captured using a number of technologies, including ultrasound images, CT scan images, X-ray images, X-ray fluoroscopy images and similar. In an image based embodiment of the method, multiple 2-d images through the body part are captured from which a 3-d image of the body part can be reconstructed using methods known in the art. Step 102 is optional and in an image-free embodiment of the method either no images are captured or any images captured are used for other purposes during the overall method, but are not actually used during the registration method of the invention.

Step 104 is also an optional step during which the medical practitioner can use a surgical planning software application to produce a plan for use by the computer aided surgery system in guiding the surgeon while carrying out the surgical procedure. In an orthopaedic surgery embodiment, the planning software can include routines, methods and procedures for selecting a orthopaedic implant, selecting the size of an implant and selected the intended implantation position for an implant.

Step 106 relates to registering the position of the body part of the patient with the computer aided surgery system. In general terms, the body part has a position in a real world reference frame, or co-ordinate system, and the computer aided surgery system has its own reference frame, or co-ordinate system. Registration, in general, refers to mapping the position of the body part in the real world into the reference frame of the computer aided surgery system. Registration can then allow, for example, images of the body part used by the computer aided surgery system to be registered with the body part position so as to provide image guided surgery functionalities and/or to allow the surgical plan generated by the surgical planning application to be registered with the body part position.

Step 108 generally corresponds to carrying out a computer aided surgical procedure on the body part using the computer aided surgery system. The computer aided surgery system can provide image guided surgery functionalities to guide and assist the surgeon in the accurate execution of various actions during an operation, such as the making of incision and cuts, the preparation of bones for the attachment of implants and the positioning of implants. As mentioned above some of the actions carried out during the registration step 106 can be intra-operative, i.e. carried out during surgery and after an initial incision has been made so as to provide access to a surgical site. However, generally registration of the body part is completed, before the actual surgical procedure (in the described embodiment a hip replacement operation) is carried out in a computer assisted manner.

With reference to FIG. 2 there is shown a schematic block diagram of a computer aided surgery system 110 in which the method of the invention can be implemented. The computer aided surgery system 110, generally includes a tracking system 112 in communication with a computer based control system 114 which includes a computer aided surgery software application 116. The computer aided surgery system can include a display device 118 and a database 120 for storing images and other data used by the system.

The computer aided surgery software application can include various functionalities as illustrated by a workflow module 122, surgical planning module 124 and registration module 126. The workflow module provides guidance to the surgeon of the steps to be carried out in executing the surgical procedure and generally controls and organises the surgical procedure. The planning module can be used by the surgeon to generate a surgical plan as described above. The registration module can be used by the surgeon to register the body part as will be described in greater detail below. The representation of the computer aided surgery application 116 in modular form is merely to aid explanation of the different functionalities provided and is not intended to limit the invention to the specific structure illustrated.

The tracking system 112 generally can track the positions of suitably marked tools, instruments, implants, body parts and other entities used by, manipulated or otherwise handled by the surgeon in order to carry out the computer aided surgical procedure. The tracking system generates data representing the location and/or orientation of a tracked item within the reference frame of the computer aided surgery system. The location data (e.g. co-ordinates in a 3-d space) and orientation data (e.g. pitch, roll and yaw) will generally be referred to as position data.

In a preferred embodiment, the tracking system is a wireless tracking system which determines the position of a marked item within an electromagnetic field. An item, e.g. pointer 128, has a handle and a probe with a tip at a free end. The handle of the pointer includes a marker in the form of three mutually perpendicular sensor coils. The sensor coils can measure the components of the electromagnetic field generated by field generating coils (not shown) of the tracking system 112. The filed generating coils generate a time varying magnetic field distribution. A time varying magnetic field will also have corresponding electric field components and hence the field is generally an electromagnetic field. However, the electromagnetic field will generally be referred to as a magnetic field as it is principally the magnetic field components that are sensed by sensor coils in the marker by induction.

The marker includes signal processing circuitry and an antenna by which the marker can wirelessly communicate with the tracking system using a radio frequency signal over communication channel 130. The marker can include an on board power source, such as a battery. Alternatively, the marker can include a power coil by which the marker is inductively powered by a further electromagnetic power signal generated by the tracking system. The marker can also transit an identifier data item to the tracking system so that the computer aided surgery system can identify the entity with which the received position data is associated. The computer aided surgery system stores data relating to the pointer tool 128 indicating the position of the tip of the probe relative to the pointer so that the computer aided surgery system can determine the position of the tip from the position data generated as the tool is tracked.

The invention is not limited to the above described tracking system and any suitable tracking system can be used. For example a wire based rather than a wireless tracking system can be used. Different types of wireless tracking system can be used. For example acoustic based, optical based, infrared based or other electromagnetic signal based technologies can be used. A suitable infrared based system, using passive markers which reflect infrared radiation, is the Vector Vision system as provided by BrainLAB AG of Heimstetten, Germany.

Although the tracking and computer control system are illustrated separately, it will be appreciated that in practice the tracking system and computer control system can be integrated into a single system, or into a greater number of sub systems, and the invention is not intended to be limited to the specific structure shown.

FIG. 2 also shows a proximal part of a femur 130 of a patient. Use of the computer aided surgery system 110 in the registration of a femur will now be described in greater detail with reference to FIGS. 3 and 4. FIG. 3 shows a flow chart illustrating a general surgical method 140 carried out by a surgeon, or other medical practitioner, so as to register a body part with a computer aided surgery system. FIG. 4 shows a flowchart illustrating a data processing method 160 carried out by the registration software application 126 being executed by the computer aided surgery system so as to provide the registration method. A hip replacement operation will be described below by way of example only.

A percutaneous method can be used in which the surgeon palpates the body part and identifies landmark points and surface points of the underlying bone through the skin. A subcutaneous method can also be used in which the surgeon identifies landmark points of the bone directly through incisions or other openings at the surgical site, or elsewhere, that provide access to the bone. The later is preferred as it provides a more accurate registration of the bone position. However, in some circumstances a percutaneous approach can be preferred, for example, if the skin is sufficiently thin, if the bone has significant landmark and surface features, e.g. the pelvis, or as part of a pre-operative procedure. A subcutaneous embodiment will be described below.

At step 142, the surgeon attaches a marker which is trackable by the tracking system to the femur via an incision. The marker allows the computer aided surgery system to monitor the current position and orientation of the femur as the surgeon moves and manipulates the bone during surgery.

At step 144, the surgeon uses a marked pointer instrument to identify certain landmark points of the bone which can be used to define the bones general shape, size and direction. In particular, the surgeon identifies at least one landmark point toward a proximal end of the femur, which in this embodiment is toward the pelvis. The surgeon also identifies at least one landmark point toward a distal end of the femur, which in this embodiment is toward the knee. The surgeon can place the tip of the pointer on the left and right epicondyles of the knee to identify two distal landmarks. The surgeon can place the tip of the pointer on the greater trochanter so as to identify the proximal landmark point. Other points on the femur can be used for the proximal landmark point, such as the top of the femur, the lesser trochanter, the piriformis fossa and the tubercle.

In some instances, access to a suitable landmark may not be available, in which case a different non-tactile approach to identifying landmarks can be used. For example, a motion analysis procedure can be used to identify the centre of rotation of the hip joint which corresponds closely to the centre of the femoral head which can be used as the proximal landmark point. In this approach, the femur is articulated over a solid angle and moved over a generally cone shaped space while the position of the marker attached to the bone at step 142 is tracked. From the locus of the surface traced by the marker it is possible to identify a fixed point corresponding to the centre of motion which is the position of the centre of the femoral head.

After the landmark points have been acquire, at step 146 the surgeon places the tip of the pointer 128 at a plurality of positions 132 on the surface of the femur 130, as illustrated in FIG. 2. The plurality of positions typically extend over a substantial area of the surface of the bone and over an anatomical feature of the bone having a complex or characteristic shape. A reasonable number of points, for example 20 or so, are acquired so as to provide a mesh or net extending over the surface of the bone and the anatomical feature.

A fit of the acquired bone points to a representation of the bone is then carried out by the computer aided surgery system as will be described in greater detail below. Then at step 148 a fitted image of the bone is displayed to the surgeon overlaid with a plurality of visual indicia each corresponding to a one of the plurality of acquired points, within the reference frame of the tracking system. Each of the visual indicia, e.g. a cross or dot, is presented so as to visually represent the closeness of the point to the fitted position of the bone.

For example, different shaped indicia can be used to indicate the closeness of fit. Different sized indicia can be used to indicate the closeness of fit, e.g. the closer a point to the fit, the smaller the indicium. Different levels of transparency can be used to indicate the closeness of fit, e.g. the further a point to the fit the fainter the indicium. Different shades of a colour can be used to indicate the closeness of fit, e.g. a gray scale in which the closer that point to the fit the whiter the indicium. Different colours can be used to indicate the closeness of fit, e.g. a spectrum of colours can be used with points close to the fit being coloured toward one end of the spectrum and points toward the other end being coloured toward the other end. Instead of using a continuum or range of values, a discrete system can be used in which a point is assigned to one of a number of classes. For example points close to the fit can be coloured green, points far from the fit can be coloured red and points neither close nor far can be coloured amber. Alternatively a binary scheme can be adopted, in which points close to the fit are coloured green and points far from the fit are coloured red. In another scheme, only indicia corresponding to points far from the fit are displayed and points determined to be sufficiently close to the fit are not displayed.

Irrespective of the display scheme used, the surgeon is presented with a display of an image of the bone together with an indication of the closeness of the captured points to the fitted position of the bone. The surgeon reviews the displayed fit and can decide whether the fit is acceptable. From the displayed fit and position indicia, the surgeon may determine that it appears that a one of the landmark points was incorrectly identified and so at step 150 the surgeon can select to re-acquire a one or more of the landmark points and all the points and bone representation can be re-fitted and the re-fitted bone image and points can be re-displayed. Alternatively, or additionally, the surgeon may determine that a one or more of the surface points may have been incorrectly identified and so at step 152 the surgeon can select to re-acquire a sub set of one or more of the surface points and all the points and bone representation can be re-fitted and the re-fitted bone image and points can be re-displayed. In one embodiment, the computer program automatically identifies any specific points that should be re-acquired, either landmark or surface points, for example by displaying them coloured red, so that the surgeon does not have to carry out any assessment of the fit. Hence, the computer program automatically guides the surgeon to re-acquire only those points which were likely acquired in error, rather than having to re-acquire all points.

Alternatively, or additionally, the surgeon may determined that a one or more of the acquired points, surface or landmark, may not be useful in achieving a good fit and so at step 156 the surgeon can select to remove points from the set of points being used for the fit and the remaining points and bone representation can be re-fitted and the re-fitted bone image and points can be re-displayed.

The re-acquisition of landmark and/or surface points and/or the removal of points from the points being fitted can be repeated iteratively until a fit that the surgeon is happy with has been achieved. The method then ends with the bone having been accurately registered in the reference frame of the computer aided surgery system so that the computer aided surgical procedure can proceed.

It will be appreciated that the above described method improves the efficiency and accuracy with which registration can be carried out. Firstly, the surgeon is presented with an indication of the closeness of fit either for each of the captured points or for those captured points determined to be not sufficiently close. Hence the surgeon is better informed as to which of the points may be responsible for a poor fit of the points to the bone representation and provides a more interactive method for the surgeon. Further, the surgeon can select to recapture only a single or a few points and not all of the points. Hence, if the fit is not acceptable, the surgeon does not need to recapture all of the points used for a fit and so the speed of carrying out a reliable registration procedure is increased.

With reference to FIG. 4 there is shown a process flow chart illustrating a process 160 carried out by the registration software application 126 used by the surgeon in method 140. At step 162, the process captures and stores the landmark positions derived from the tracking data 164 from the tracking system, for each of the landmarks identified by the surgeon, e.g. the centre of the femoral head and the right and left epicondyles. Then at step 166, the process captures and stores the positions of the plurality of points identified on the surface of the bone by the surgeon, from the tracking data provided by the tracking system. The plurality of points provides a net or mesh defining the shape of the surface of the bone in their locale.

Then at step 168 a representation of the femur is fitted to the position data. Either a patient scan based approach or a no patient scan based approach can be used. If a scan of the patient's bone has been carried out, pre-operatively or intra-operatively, then the images of the patients bone obtained from the scan can be fitted to the set of positions at step 168. If no scan of the patient's bone is available then in a patient scan free approach, a slightly different approach is used. Database 120 stores a number of scans of femurs from which a 3-d image or model of each femur can be generated. For example the scans for 30 or so femurs of various sizes and geometries are stored in the database. Hence, rather than using a scan of the patient's femur in fitting step 168, a model or virtual femur derived from one of the femur scans stored in the database is used instead in the fitting step 168.

FIG. 5 shows a flow chart illustrating the process 190 of fitting an image or representation of the bone to the position data in greater detail, and corresponding generally to step 168 in FIG. 4.

In a patient scan free approach, at step 192, the separation between the distal and proximal landmark points is used to determine a measure of the size of the femur. The database is then searched to find the stored femur scan most closely matching the size of the patient's femur. This can help the fitting process as it has been found that there tends to be a relationship between the size of a femur and its other geometric properties, such as its thickness. Then at step 194, as the positions of the points corresponding to the proximal and distal end are known in the reference frame of the tracking system, the general direction of the femur can be determined. Also, using, for example, the positions of the left and right epicondyle landmark points, the general orientation of the femur can also be determined and so the overall position of the femur in the reference frame of the tracking system are known. At step 196, the 3-d image of the femur can be scaled up or down using the landmark points so that the 3-d image of the femur more closely matches the size of the patient's femur. Then at step 198 the 3d image or model of the femur is fitted to the set of surface data points using a fitting algorithm, such as a least squares fitting algorithm.

If a scan of the patient's femur is available, then instead of using a model femur derived from a scan of another femur, a model or 3-d image derived from the patient's scan is used instead in the fitting process. Hence, a number of the steps shown in FIG. 5 can be omitted. For example steps 192 and 196 can be omitted. However, in embodiments using X-rays, X-ray images can be magnified and therefore a step equivalent to step 192 which determines the actual size of the patients femur is required in order scale the x-ray image to the actual size of the patient's femur. The direction and orientation are determined at step 194 using the landmark points and then at step 198 a least squares fit of the model of the patient's femur to the set of surface data points is carried out. The landmark points can also be used to improve the fit.

A quantitative measure or metric of the overall closeness of the fit can also be generated from the fitting algorithm, such as the average closeness metric of the points or a chi-squared value for the least squares fit.

Process flow then proceeds at step 170 of FIG. 4. After the fit has been carried out some quantitative measure or metric is obtained at step 170 from the fitting procedure 190 which provides an indication, for each of the landmark and surface points, how close they point is to the fitted representation of the femur (whether derived from the patients scan or from a morphed scan derived obtained from the database). For example, the closeness or error metric can be the magnitude of the distance between each point and the fitted surface of the femur. Then at step 172 the image of the femur as fitted in the reference frame of the tracking system is displayed and an indicia is also displayed for the points at their respective positions in the reference frame of the tracking system. Each point can have the closeness of their fit to the image encoded in a visualisable manner as discussed above.

For example, the closeness of a point to the fitted image can be indicated by the colour that the point is displayed in. A gray scale or a colour scale can be used and the error or closeness metric can be used to look up the appropriate colour in which to display the point. Alternatively, a binning approach can be used in which thresholds are used to discriminate between points so that points having an closeness metric falling within a certain range are displayed in one colour (e.g. points less than 2 mm distant can be displayed in green) and points having a closeness metric falling within another range are displayed in another colour (e.g. points more than 2 mm distant can be displayed in red). In an alternate approach only those points having a closeness metric exceeding a threshold value can be displayed, e.g. only those more then 2 mm distant, so that the surgeon can readily identify those points possibly leading to a poor fit.

After displaying the fitted image of the femur and the points at step 172, the process can determine whether an automatic fitting correction procedure should be carried out at step 174. This can be determined automatically by the program, for example by comparing the overall fit metric with a threshold value to determine whether the fit can be considered acceptable or not, or manually, by the surgeon entering a command requesting the program to try and determine what point or points may be causing a poor fit with the femur image data.

Responsive to either a command or an automatic determination that an auto-correction procedure is to be carried out, process flow branches and an auto-correction procedure is carried out at step 176. FIG. 6 shows a flow chart illustrating the process 200 of automatically determining a point to correct or re-acquire in order to improve the fit in greater detail, and corresponding generally to step 176 in FIG. 4. Process 200 is based on identifying any systematic variations in the fit metrics which may indicate that a poor fit may be the result of an unrepresentative point, or otherwise incorrectly captured point, rather than merely as a result of the random variation in the capture of points or in the shape of a body part.

The process 200 begins at step 202 in which the set of error metrics for the set of captured surface points is analysed by assessing the spatial variation in the error metrics. For example there may be a systematic increase in the value of error metrics in a certain direction, e.g. toward the distal part of the femur. This could indicate that a one or both of the distal landmark points was incorrectly captured. One method for assessing the spatial variation would be to look at the gradient of the error metrics in certain directions, e.g. in the proximal-distal directions. If variations in the error metrics are random, then the gradient should tend toward zero or be very low. However, if there is an increasing, or decreasing, error metric then a significant non-zero gradient would exist and can be used in the assessment of the spatial distribution.

After the spatial distribution has been assessed, the assessment is used in step 204 to identify the captured point or points which are likely to be responsible for the poor fit. For example, a non-zero error metric gradient in the proximal to distal direction may indicate that the distal landmark was incorrectly captured. Therefore step 204 uses the assessment, or some characteristic property, of the spatial distribution of the error metrics to identify a likely incorrectly captured point. Then at step 206 the likely responsible point or points can be highlighted on the screen display and/or flagged for further processing.

Returning to FIG. 4, process flow returns to step 162 and/or 166 and the process prompts the surgeon to re-identify the point using the marked instrument and the process captures the position data for the re-captured point using the tracking data 164 from the tracking system. Processing continues as described previously and a further fit is carried out using with the newly captured point being used in place of the originally captured point. The surgeon can enter a command to over ride the prompt to re-capture the point if from a review of the display it appears that the result of assessment method 200 is incorrect.

After any auto-identification of points possibly requiring correction at step 174, the users is prompted to identify any points that they consider to be incorrectly captured, or causing a poor fit, from the displayed points. For example a particular surface point may correspond to an unusual anatomic feature particular to the patient and which is resulting in a poor fit to a model femur. This point will have a high error metric and will be clearly identified in a visual manner. A command manually identifying the point or points is received by the process and process flow returns to step 162 and/or 166 at which the surgeon can re-identify the point or points, as described above, and a further fit is carried out using the newly captured point positions and the originally captured point positions.

After any manual corrections to the set of points have been carried out, process flow proceeds to step 180 at which it is determined whether any points are to be removed from the set of position data used in the fit. A command is received identifying any points to be removed and process flow proceeds to step 168 at which the fit is carried out again but using a reduced set of position data. In this way, the surgeon can identify any individual points which are considered not to be useful in providing a good fit (for example, as not being on the bone or not being a part of the bone surface being fitted, e.g. osteophytes), and the removal of which from the position data may lead to a more accurate fit of the remaining position data. Hence it is not necessary to re-capture the positions of points which are not considered to be contributing to a valid fit.

When a good fit has been achieved, the model or image of the femur is considered to have been registered in the reference frame of the tracking system at step 182 and the registration process 160 terminates.

Generally, embodiments of the present invention, and in particular the processes involved in registration of the body part employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media, computer program code or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

FIG. 7 illustrates a typical computer system that, when appropriately configured or designed, can serve as a part of the computer aided surgery system of this invention. The computer system 500 includes any number of processors 502 (also referred to as central processing units, or CPUs) that are coupled to storage devices including primary storage 506 (typically a random access memory, or RAM), primary storage 504 (typically a read only memory, or ROM). CPU 502 may be of various types including microcontrollers and microprocessors such as programmable devices (e.g., CPLDs and FPGAs) and unprogrammable devices such as gate array ASICs or general purpose microprocessors. As is well known in the art, primary storage 504 acts to transfer data and instructions uni-directionally to the CPU and primary storage 506 is used typically to transfer data and instructions in a bi-directional manner. Both of these primary storage devices may include any suitable computer-readable media such as those described above. A mass storage device 508 is also coupled bi-directionally to CPU 502 and provides additional data storage capacity and may include any of the computer-readable media described above. Mass storage device 508 may be used to store programs, data and the like and is typically a secondary storage medium such as a hard disk. It will be appreciated that the information retained within the mass storage device 508, may, in appropriate cases, be incorporated in standard fashion as part of primary storage 506 as virtual memory. A specific mass storage device such as a CD-ROM 514 may also pass data uni-directionally to the CPU.

CPU 502 is also coupled to an interface 510 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 502 optionally may be coupled to an external device such as a database or a computer or telecommunications network using an external connection as shown generally at 512. With such a connection, it is contemplated that the CPU might receive information from the network, or might output information to the network in the course of performing the method steps described herein.

Although the above has generally described the present invention according to specific processes and apparatus, the present invention has a much broader range of applicability. In particular, aspects of the present invention is not limited to any particular bone or surgical procedure and can be applied to any surgical procedure in which it is useful to be able to register a body part with a reference frame of a computer aided surgery system. One of ordinary skill in the art would recognize other variants, modifications and alternatives in light of the foregoing discussion.

1. A computer implemented method for registering the position of a body part of a patient with a computer aided surgery system, comprising:
determining the position of a plurality of points of the body part;
fitting the plurality of points to a representation of the body part;
determining a closeness of fit for each of the plurality of points; and
displaying an indication of the closeness of fit for at least one of the plurality of points.

2. A method as claimed in claim 1, wherein displaying an indication of the closeness of fit includes displaying the indication at the determined position of the point overlaid on an image of the body part at a fitted position of the image of the body part.

3. A method as claimed in claim 1 or 2, where the indication is a visual indication.

4. A method as claimed in claim 3, wherein the visual indication is colour.

5. A method as claim in claim 4, wherein the colour varies depending on the closeness of fit.

6. A method as claimed in any preceding claim, wherein an indication of the closeness of fit is displayed for each of the plurality of points.

7. A method as claimed in any preceding claim and further comprising:
re-determining the position of at least a one of the plurality of points; and
re-fitting those points of the plurality of points whose position has not been re-determined and those points whose position has been re-determined.

8. A method as claimed in any preceding claim, wherein the plurality of points includes a set of landmark points and a set of surface points.

9. A method as claimed in claim 8, wherein the positions of the landmark points are used to determine the direction and/or the size and/or the orientation of the body part.

10. A method as claimed in claim 8 or 9, wherein the positions of the surface points are used to fit the positions of the plurality of points to the representation of the body part.

11. A method as claimed in any preceding claim, wherein the representation of the body part is a captured image of the body part or derived from a captured image of the body part.

12. A method as claimed in any of claims 1 to 10, wherein the representation of the body part is a virtual model of the body part or derived from a virtual model of the body part.

13. A method as claimed in any preceding claim and further comprising assessing the spatial distribution of the closeness of fit of the plurality of points to identify at least a one of the plurality of points causing an unsuccessful fit.

14. A method as claimed in any preceding claim, wherein one of the plurality of points is a landmark point, and wherein the closeness of fit of the landmark point is determined, and if the closeness of fit of the landmark point exceeds a threshold, then displaying a visual indication to recapture the landmark point.

15. A method as claimed in claim 14, and further comprising:
re-determining the position of the landmark point; and
re-fitting using the landmark point whose position has been re-determined.

16. A computer aided surgery system for registering the position of a body part of a patient comprising:
a tracking system for determining the position of an instrument engageable with a plurality of points of the body part; and
a control system configured to:
determine the position of the plurality of points of the body part;
fit the plurality of points to a representation of the body part;
determine a closeness of fit for each of the plurality of points; and
display an indication of the closeness of fit for at least one of the plurality of points on a display device.

17. A method for registering the position of a body part of a patient with a computer aided surgery system, comprising:
identifying the position of a plurality of points of the body part with an instrument trackable by a tracking system;
determining whether to re-identify the position of at least a one of the plurality of points from a display of an indication of the closeness of fit for at least one of the plurality of points, the closeness of fit having been determined for each of the plurality of points from a fit of the plurality of points to a representation of the body part; and
re-identifying the position of a subset of the plurality of points of the body part, the subset comprising fewer points than all of the plurality of points.

18. Computer program code executable by a data processing device to provide the method of any of claims 1 to 15 or the system of claim 16.

19. A computer program product comprising a computer readable medium bearing computer program code as claimed in claim 18.
